(19) United States
(12) Patent Application Publication
(10) Pub. No.: US 2005/0020468 A1
(43) Pub. Date: Jan. 27, 2005

(54) New branched sulfates for use in personal care formulations

(76) Inventors: Seren Frantz, Bensalem, PA (US); Stewart Alexander Warburton, West Windsor, NJ (US)

Correspondence Address: KEVIN E. MC VEIGH;RHODIA INC., 259 PROSPECT PLAINS ROAD, CRANBURY, NJ, 08512, US

(21) Appl. No.: 10/896,464

(22) Filed: Jul. 22, 2004

Related U.S. Application Data
(60) Provisional application No. 60/489,314, filed on Jul. 22, 2003.

(51) Int. Cl. C11D 17/00

(52) U.S. Cl. 510/424

(57) ABSTRACT

A structured surfactant composition containing one or more branched alkyl (ether) sulfates according to the formula: RO(CH₂CH₂O)ₙSO₃M, wherein R is branched (C₈-C₁₈)alkyl or branched (C₈-C₁₈)alkenyl, n has an average value of from 0 to about 7 and M is a solubilizing cation, provided that M cannot be sodium if n is greater than or equal to 1, and a structurant, wherein the composition exhibits non-Newtonian shear thinning viscosity and is capable of suspending insoluble or partially insoluble components

### FIELD OF THE INVENTION

This invention relates to use of branched alkyl (ether) sulfates, that is, branched alkyl sulfates and/or alkyl ether sulfates, in personal care formulations such as body washes, shampoos, baby cleansing products, facial cleansers, hand soaps, and skin cleansers.

### BACKGROUND OF THE INVENTION

The only branched alkyl (ether) sulfate currently used in personal care formulations is the sodium trideceth-3 sulfate, which is used primarily in baby products, facial washes, and occasionally in suspending formulations. U.S. Pat. No. 6,150,312 discloses that branched (C₁₀-C₂₂)alkyl alkali metal sulfates provide enhanced freeze thaw stability in structured liquid compositions, but appears to provide actual examples of only sodium tricedeth (sic) sulfate.

### SUMMARY OF THE INVENTION

The present invention is directed to an aqueous structured surfactant composition, comprising:
one or more branched alkyl (ether) sulfates according to the formula (1):
   RO(CH₂CH₂O)ₙSO₃M (1)
   wherein:
R is branched (C₈-C₁₈)alkyl or branched (C₈-C₁₈)alkenyl,
n has an average value of from 0 to about 7 and
M is a solubilizing cation, provided that M cannot be sodium if n is greater than or equal to 1, and
   a structurant,
   wherein the composition exhibits non-Newtonian shear thinning viscosity and is capable of suspending insoluble or partially insoluble components.

### DETAILED DESCRIPTION OF INVENTION AND PREFERRED EMBODIMENTS

The branched alkyl (ether) sulfates of interest include, for example, branched alkyl (ether) sulfates with a low level of alkoxylation or with no alkoxylation, such as but not limited to sodium tridecyl sulfate or ammonium tridecyl sulfate, as well as branched alkyl (ether) sulfates with cations other than sodium, such as for example, ammonium trideceth sulfate.

The aqueous composition of the present invention further comprises water, and may, optionally further comprise other surfactants (such as other anionic surfactants, cationic surfactants, amphoteric surfactants, or nonionic surfactants), thickeners, benefit agents, electrolytes, fragrances, dyes, preservatives, or other common ingredients used in personal care formulations. Of particular interest is the use of these branched alkyl (ether) sulfates in non-Newtonian shear thinning formulations capable of suspending insoluble or partially insoluble components. The non-Newtonian shear thinning formulations capable of suspending insoluble or partially insoluble components are formed by including water, a structurant, and optionally other surfactants including anionic surfactants, nonionic surfactants, amphoteric surfactants and cationic surfactants, or any combination of the above.

In one embodiment, the composition of the present invention comprises one or more branched alkyl (ether) sulfates according to formula (1), wherein is R is a branched (C₁₂-C₁₈)alkyl or branched (C₁₂-C₁₈)alkenyl, more typically branched (C₁₂-C₁₈)alkyl. In one embodiment, R comprises one or more branched (C₁₃)alkyl groups. As used herein in reference to an organic moiety, the notation (Cₙ-Cₘ), wherein n and m are each positive integers, means that the moiety contains from n to m carbon atoms per moiety.

In one embodiment, the composition of the present invention comprises one or more branched alkyl (ether) sulfates according to formula (1), wherein n is from 0 to 3. In another embodiment, n is from 0 to less than 1. In another embodiment, n is about 0. In yet another embodiment, n is between 0 and 7, more typically between 0 and 3, and even more typically between 0 and 1.

In one embodiment, the composition of the present invention comprises one or more branched alkyl (ether) sulfates according to formula (1), wherein M is selected from sodium, magnesium, potassium, ammonium, and substituted ammonium. As sued herein, “substituted ammonium” means an ammonium ion in which from one to three H substituents of an ammonium ion are replaced by organic groups, typically (C₁-C₄)alkoxyl groups, such as for example, monoethoxyl ammonium, diethoxyl ammonium, and triethoxyl ammonium. In another embodiment, M is selected from magnesium, potassium, ammonium, and substituted ammonium, more typically, ammonium or substituted ammonium, and still more typically, ammonium.

In one embodiment, the composition of the present invention comprises, on the basis of 100 parts by weight (“pbw”) of the composition, from about 3 to about 50 pbw, more typically from about 8 to about 40 pbw, still more typically from about 10 to about 20 pbw, of one or more branched alkyl (ether) sulfates according to formula (1).

Structurants are used in combination with anionic surfactants, such as the branched alkyl sulfate or alkyl ether sulfate of the composition of the present invention, to produce the desired suspending properties. Suitable structurants include electrolytes, cationic surfactants, and nonionic surfactants, as well as mixtures thereof. Suitable cationic surfactants and nonionic surfactants are described in more detail below. In one embodiment, the structurant comprises a nonionic surfactant selected from fatty alcohols, fatty acids, fatty acid esters, and alkanolamides.

An effective amount of structurant is an amount that is at least equal to the amount required to provide, in combination with the branched alkyl (ether sulfate component (as well as any added optional anionic surfactant) of the composition of the present invention, the desired non-Newtonian shear thinning viscosity and suspending properties, typically from about 0.1 to about 20 pbw, more typically from about 0.5 pbw to about 10, and still more typically from about 1 to about 5 pbw, of the structurant per 100 pbw of the structured surfactant composition.

The electrolyte can be added separately to the composition or it can be included as part of one of the other raw materials. The electrolyte preferably includes an anion comprising phosphate, chloride, sulfate or citrate and a cation comprising sodium, ammonium, potassium, magnesium or mixtures thereof. Some preferred electrolytes are sodium or ammonium chloride and sodium or ammonium sulfate.

If present, the electrolyte should be present in an amount which facilitates formation of the free flowing composition. This amount will typically be from about 0.1% by weight to about 15% by weight, preferably from about 1% to about 6% by weight, but may be varied if required.

The composition of the present invention may optionally further comprise other surfactants in addition to the branched alkyl (ether) sulfate and structurant of the composition of the present invention. Such other surfactants may comprise one or more surfactants selected from other anionic surfactants in addition to the alkyl sulfate or alkyl ether sulfate, nonionic surfactants, amphoteric surfactants, zwitterionic surfactants, and cationic surfactants.

Other suitable anionic surfactants include, for example, linear alkyl (ether)sulfates, such as linear alkyl ether sulfates according to formula (1), wherein R is a linear alkyl or alkenyl having 8 to 18 carbons, typically 12 to 18 carbons, n has an average value typically between 0 and 7, preferably between 0 and 3, and M is a solubilizing cation, such as sodium, magnesium, potassium, ammonium or substituted ammonium.

Other suitable anionic surfactants include, for example, aliphatic sulfonates, such as a primary alkane (e.g., C₈-C₂₂) sulfonates, primary alkane (e.g., C₈-C₂₂) disulfonates, (C₈-C₂₂)alkene sulfonates, C₈-C₂₂ hydroxyalkane sulfonates, alkyl glyceryl ether sulfonates (AGS), aromatic sulfonates such as alkyl benzene sulfonates,

Other suitable anionic surfactants include alkyl sulfosuccinates (including mono- and dialkyl, e.g., (C₆-C₂₂)sulfosuccinates), alkyl and acyl taurates, alkyl and acyl sarcosinates, sulfoacetates, (C₈-C₂₂)alkyl phosphates, alkyl phosphate esters, alkoxyl alkyl phosphate esters, acyl lactates, C₈-C₂₂ monoalkyl succinates and maleates, and acyl isethionates. Sulfosuccinates may include monoalkyl sulfosuccinates having the formula:
R₄O₂CCH₂CH(SO₃M)CO₂M, (2)
amido-MEA (monoethanolamide) sulfosuccinates of the formula
R₄CONHCH₂CH₂O₂CCH(SO₃M)CH₂CO₂M (3)
wherein R₄ ranges from C₈ to C₂₂ alkyl and M is a solubilizing cation, and amido-MIPA (monoisopropanolamide) sulfosuccinates of the formula
RCONHCH₂CH(CH₃)O₂CCH (SO₃M)CH₂CO₂M (4)
where M is as defined above for formula (ii) and R ranges from C₈ to C₂₂ alkyl.

Other suitable anionic surfactants include alkoxylated citrate sulfosuccinates and alkoxylated sulfosuccinates such as the following:
R—O—(CH₂CH₂O)ₙC—CH₂CH(SO₃M)CO₂M (5)
where M is as defined above for formula (ii) and R ranges from C₁₀ to C₂₂ alkyl.

Sarcosinates are generally indicated by the formula
RCON(CH₃)CH₂CO₂M (6)
wherein R ranges from C₈ to C₂₂ alkyl and M is a solubilizing cation.

Taurates are generally identified by the formula:
R₂CONR₃CH₂CH₂SO₃M (7)
wherein R₂ ranges from C₈ to C₂₂ alkyl, R₃ ranges from C₁ to C₄ alkyl, and M is a solubilizing cation.

Other suitable anionic surfactants include carboxylates of the following formula:
R—O—(CH₂CH₂O)ₙCO₂M (8)
wherein R is C₈ to C₂₂ alkyl, n is 0 to 20, and M is as defined above in formula (ii). Other carboxylates which can be used include amido alkyl polypeptide carboxylates.

Other suitable anionic surfactants include (C₈-C₂₂)acyl isethionates. These esters are prepared by reaction of alkali metal isethionate with mixed aliphatic fatty acids having from about 6 to about 22 carbon atoms and an iodine value of less than about 20. At least about 75% of the mixed fatty acids have from about 12 to about 18 carbon atoms and up to about 25% have from about 6 to about 10 carbon atoms.

The acyl isethionates include alkoxylated isethionates such as those described in Ilardi et al., U.S. Pat. No. 5,393,466, hereby incorporated by reference to the extent it is consistent with this invention and application, according to the general formula: 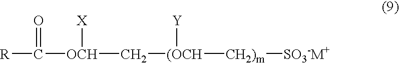
wherein R is an alkyl group having 8 to 22 carbons, m is an integer from 1 to 4, X and Y are hydrogen or an alkyl group having 1 to 4 carbons, and M⁺ is a monovalent cation such as, for example, sodium, potassium or ammonium.

The amount of anionic surfactant ingredient is typically about 5% to about 30%, and preferably about 10% to about 20% by weight of the composition.

Except in the examples or where otherwise explicitly indicated, all numbers in this disclosure indicating amounts or ratios of materials or conditions of reactions, physical properties of materials and/or use are understood to be modified by the word “about”.

Where weight of a surfactant is utilized in this disclosure, weight is understood to mean weight of active surfactant, with the exception of the examples in the tables.

Cationic surfactants are described as carrying a positive charge, usually on a nitrogen atom in the form of an amine salt or a quaternary ammonium compound, and include monoalkyl amine derivatives, dialkyl amine derivatives, or imidazoline derivatives.

Suitable cationic surfactants include compounds according to the general formula: 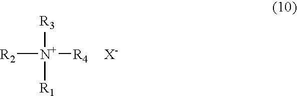
wherein the four R groups, R₁, R₂, R₃ and R₄, are hydrogen, an organic group, or a combination thereof, with the proviso that at least one of the R groups is not hydrogen. X represents a typical anion, which can include chloride, bromide, methosulfate, ethosulfate, lactate, saccharinate, acetate or phosphate. If one to three of the R groups is hydrogen, the compound may be referred to as an amine salt. Some examples of cationic amines include polyethoxylated (2) oleyl/stearyl amine, ethoxylated tallow amine, cocoalkylamine, oleylamine, and tallow alkyl amine. For quaternary ammonium compounds (generally referred to as quats) R₁, R₂, R₃, and R₄ may be the same or different, but may not be hydrogen. In one embodiment, R₁, R₂, R₃, and R₄ are (C₈-C₂₄) branched or linear, saturated or unsaturated aliphatic chains, which may comprise additional functionality such as, for example, fatty acids or derivatives thereof, including esters of fatty acids and fatty acids with alkoxylated groups, alkyl amido groups, aromatic rings, heterocyclic rings, phosphate groups, epoxy groups, and hydroxyl groups. The nitrogen atom may also be part of a heterocyclic or aromatic ring system, e.g., cetethyl morpholinium ethosulfate or steapyrium chloride. See International Cosmetic Ingredient Dictionary and Handbook, eighth edition, 2000, Volume 2, p. 1703.

Suitable quaternary ammonium compounds of the monoalkyl amine derivative type include, for example:
cetyl trimethyl ammonium bromide, also known as CETAB or cetrimonium bromide 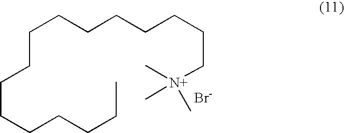
cetyl trimethyl ammonium chloride, also known as cetrimonium chloride 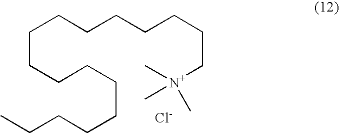
myristyl trimethyl ammonium bromide, also known as myrtrimonium bromide or Quaternium-13 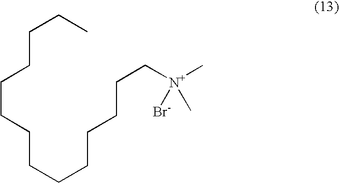
stearyl dimethyl benzyl ammonium chloride, also known as stearalkonium chloride 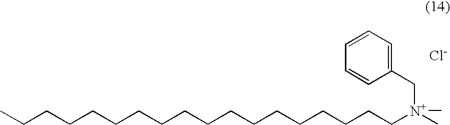
oleyl dimethyl benzyl ammonium chloride, also known as olealkonium chloride 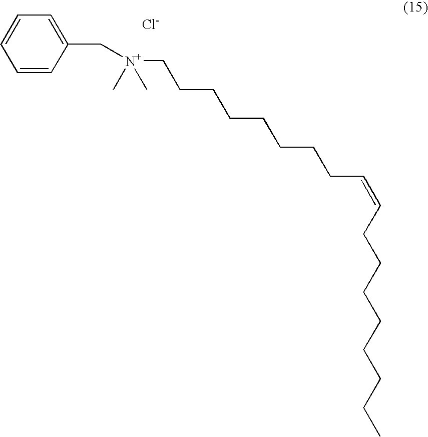
lauryl/myristryl trimethyl ammonium methosulfate, also known as cocotrimonium methosulfate 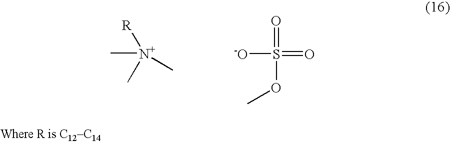
and cetyl-dimethyl-(2)hydroxyethyl ammonium dihydrogen phosphate, also known as hydroxyethyl cetyldimonium phosphate

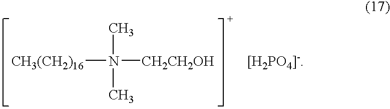

Other suitable cationic surfactants include, for example, babassuamidopropylkonium chloride, cocotrimonium chloride, distearyldimonium chloride, wheat germ-amidopropalkonium chloride, stearyl octyldimonium methosulfate, isostearaminopropal-konium chloride, dihydroxypropyl PEG-5 linoleaminium chloride, PEG-2 stearmonium chloride, Quaternium 18, Quaternium 80, Quaternium 82, Quaternium 84, behentrimonium chloride, dicetyl dimonium chloride, behentrimonium methosulfate, tallow trimonium chloride and behenamidopropyl ethyl dimonium ethosulfate.

Other suitable cationic surfactants include, for example, dialkyl amine derivatives s. These compounds include, for example, distearyldimonium chloride, dihydrogenated palmoylethyl hydroxyethylmonium methosulfate, dipalmitoylethyl hydroxyethylmonium methosulfate, dioleoylethyl hydroxyethylmonium methosulfate, and hydroxypropyl bisstearyldimonium chloride.

Other suitable cationic surfactants include, for example, quaternary ammonium compounds of the group commonly referred to as imidazoline derivatives. These compounds include, for example, isostearyl benzylimidonium chloride, cocoyl benzyl hydroxyethyl imidazolinium chloride, cocoyl hydroxyethylimidazolinium PG-chloride phosphate, Quaternium 32, and stearyl hydroxyethylimidonium chloride.

Mixtures of cationic surfactants may also be used. If present, the amount of active cationic surfactant, either from a single cationic or from multiple cationics is typically from about 0.1% to about 20%, preferably from about 1% to about 10%, and more preferably from about 2% to about 6% by weight of the composition.

Nonionic surfactants are neutral surfactants carrying no net charge.

Nonionic surfactants that are useful as structurants include alkanolamides, for example, compounds having the general structure of: 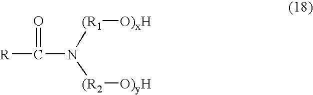
wherein R is C₈ to C₂₄, or preferably in some embodiments C₈ to C₂₂, or in other embodiments C₈ to C₁₈, saturated or unsaturated, straight chain or branched aliphatic groups, R₁ and R₂ are the same or different C₂-C₄ straight chain or branched aliphatic groups, x=0 to 10, y=1 to 10, wherein the sum of x and y is less than or equal to 10.

Suitable alkanolamides preferably have a (C₈ to C₂₄)aliphatic chain and the may include one to two alkanol groups which may either have a hydrocarbon backbone or an alkoxy backbone. The hydrocarbon alkanol groups may be (C₂-C₄) straight chain or branched aliphatic groups. The amount of alkanolamide in the composition, if present, can be 0.1% to about 10% by weight, and in some embodiments is preferably about 2% to about 5% by weight. Some preferred alkanolamides include cocamide MEA (coco monoethanolamide) and cocamide MIPA (coco monoisopropanolamide).

The term “alkanolamide” is used collectively hereinafter to include long chain aliphatic acid alkanolamides, alkoxy long-chain aliphatic acid alkanolamides, and mixtures thereof. Further, long-chain aliphatic acid alkanolamides may also be referred to in the art as fatty acid alkanolamides. Alkoxylated is taken to mean an alkanolamide derivitized with ⁻(R₁O)ₓ H wherein R₁ is a C₂ to C₄ straight chain or branched aliphatic group and x is 2 to 10.

Suitable fatty acids include, for example, saturated or unsaturated, linear or branched (C₁₀-C₂₂) acids, such as, for example, lauric acid, oleic acid, stearic acid, myristic acid, and cetearic acid, isostearic acid, linoleic acid, linolenic acid, ricinoleic acid, elaidic acid, arichidonic acid, myristoleic acid, palmitoleic acid, or the neutralized versions thereof. Ester derivatives include propylene glycol isostearate, propylene glycol oleate, glyceryl isostearate, glyceryl oleate, polyethylene glycol distearates and polyglyceryl diisostearate.

The compositions of the invention utilize about 0.1% to 15% by wt., preferably 0.5 to 10% by wt. of a fatty acid or fatty acid ester structurant.

Suitable non-ionic surface-active agents include, for example, ethyoxylated fatty alcohols and especially those derived from lauryl, cetylstearyl, stearyl, cetyl, oleol and oleocetyl alcohols. Sucroglycerides can also be used. Sucroglycerides are mixtures of compounds which are prepared by transesterification of natural or synthetic triglycerides with sucrose. These mixtures contain monoglycerides, diglycerides, small amounts of non-transesterified triglycerides, monoesters and diesters of sucrose.

EP-A-0,091,331 describes a process for preparing free-flowing sucroglycerides, and also indicates that such sucroglycerides have surface-active properties.

In one embodiment, the composition comprises from about 3 to about 50 percent by weight (wt %), more typically from about 3 to about 30 wt % of active surfactants. In another embodiment, the composition comprises from about 5 to about 50 wt %, more typically from about 8 to about 40 wt %, and still more typically from about 10 to about 25 wt %, of active surfactants. Frequently, surfactants are sold as solutions in water or other solvents which dilute them to less than 100% active surfactant, therefore the “active surfactant” means actual amount of surfactant delivered to composition from a commercial surfactant preparation.

As used herein, the terms “branching” or “branched” mean that at least one carbon atom of the aliphatic chain is joined to three or four other carbon atoms. Unsaturation means that at least two carbon atoms of the aliphatic chain are joined by a double or triple bond.

Additional surfactants from the classes of nonionic surfactants, amphoteric and/or zwitterionic surfactants, and cationic surfactants may optionally be incorporated so as to form a free flowing composition that is capable of suspending water-insoluble particles or partially insoluble components.

Amphoteric and/or zwitterionic surfactants that may be optionally included in the composition of the present invention preferably include at least one acid group, which may be a carboxylic or a sulphonic acid group. These surfactants include quaternary nitrogen and therefore are quaternary amido acids. They generally include an alkyl or alkenyl group of 7 to 18 carbon atoms and usually comply with the overall structural formula: 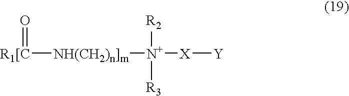
where R₁ is alkyl or alkenyl of 7 to 18 carbon atoms, R₂ and R₃ are each independently hydrogen, alkyl, hydroxyalkyl or carboxyalkyl of 1 to 3 carbon atoms, n is 2 to 4, m is 0 to 1, X is alkylene of 1 to 3 carbon atoms optionally substituted with hydroxyl, and Y is —CO₂— or —SO₃—.

Suitable amphoteric and/or zwitterionic surfactants within the above general formula include simple betaines of formula: 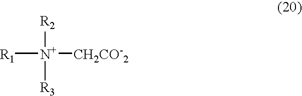
and amido betaines of formula: 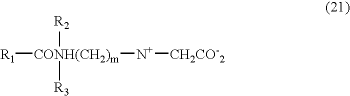
where m is 2 or 3.

In both formulae (20) and (21), R₁, R₂ and R₃ are as defined previously in connection with formula (19). R₁ may in particular be a mixture of C₁₂ and C₁₄ alkyl groups derived from coconut so that at least half, preferably at least three quarters, of the R₁ groups have 10 to 14 carbon atoms. R₂ and R₃ are preferably methyl.

A further possibility is that the amphoteric and/or zwitterionic detergent is a sulphobetaine of formula 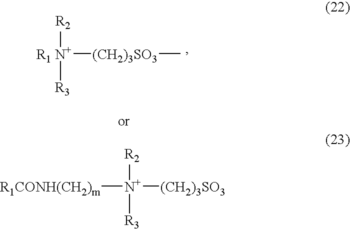
where m is 2 or 3, or variants of these in which —(CH₂)₃SO₃ is replaced by

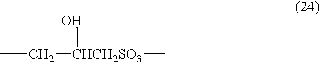

In formulas 22-24 above, R₁, R₂ and R₃ are as defined previously in connection with formula (19).

Amphoacetates and diamphoacetates may also be used. Amphoacetates generally conform to the following formula: 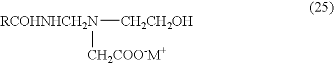
and diamphoacetates generally conform to the following formula: 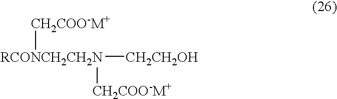
where R is an aliphatic group of 8 to 18 carbon atoms and M is a cation such as sodium, potassium, ammonium, or substituted ammonium. Sodium lauroamphoacetate, sodium cocoamphoactetate, disodium lauroamphoacetate, and disodium cocoamphodiacetate are preferred in some embodiments.

The composition of the present invention may optionally further comprise a nonionic surfactant. Nonionic surfactants which may be used include in particular the reaction products of compounds having a hydrophobic group and a reactive hydrogen atom, for example aliphatic alcohols, acids, amides and alkyl phenols, with alkylene oxides, especially ethylene oxide either alone or in combination with propylene oxide. Specific nonionic surfactant compounds include alkyl (C₆-C₂₂) phenols-ethylene oxide condensates, the condensation products of aliphatic (C₈-C₁₈) primary or secondary linear or branched alcohols with ethylene oxide, and products made by condensation of ethylene oxide with the reaction products of propylene oxide and ethylenediamine. Other so-called nonionic surfactant compounds include alkyl amine oxides, alkyl amido amine oxides, alkyl tertiary phosphine oxides, dialkyl sulphoxides, aliphatic fatty acid esters of (C₈-C₂₂) alcohols or ethoxylated alcohols, alkoxyl alkyl amines, sorbitan, sorbitan esters and sucrose esters.

The nonionic surfactant may also be a sugar amide, such as a polysaccharide amide. Specifically, the surfactant may be one of the lactobionamides described in U.S. Pat. No. 5,389,279 or one of the sugar amides described in U.S. Pat. No. 5,009,814, both of which are incorporated by reference herein to the extent that they are not inconsistent with this application.

Other surfactants which may be used are those described in U.S. Pat. No. 3,723,325, and alkyl polysaccharide nonionic surfactants as disclosed in U.S. Pat. No. 4,565,647, both of which are also incorporated by reference herein. Preferred alkyl polysaccharides are alkylpolyglycosides of the formula
R₂O(CₙH₂ₙO)ₜ(glycosyl)ₓ (27)
wherein R₂ is selected from the group consisting of alkyl, alkylphenyl, hydroxyalkyl, hydroxyalkylphenyl, and mixtures thereof, in which the alkyl groups contain from about 10 to about 18, preferably from about 12 to about 14 carbon atoms, n is from 0 to about 3, preferably 2, t is from 0 to about 10, preferably 0, and x is from about 1.3 to about 10, preferably from about 1.3 to about 2.7. The glycosyl is preferably derived from glucose. To prepare these compounds, the alcohol or alkylpolyethoxy alcohol is formed first and then reacted with glucose, or a source of glucose, to form the glucoside (attachment at the 1-position). The additional glycosyl units can then be attached between their 1-position and the preceding glycosyl unit's 2-, 3-, 4- and/or 6-position, preferably the 2-position.

In some embodiments, the preferred nonionic surfactants include alkoxy fatty acid alcohols or alkypolyglycosides. The amphoteric and/or zwitterionic surfactants preferred in some embodiments include betaines, sultaines, amphoacetates, diamphoacetates or mixtures thereof. The total amount of active nonionic surfactants and amphoteric and/or zwitterionic surfactants is typically about 1% to about 20% and preferably about 3% to about 10% by weight.

The composition of the present invention may further comprise water-insoluble particles or partially insoluble components, and/or one or more additional surfactants from the categories of anionic, nonionic, amphoteric, zwitterionic and cationic, or a combination of these.

The composition of the present invention is capable of suspending water-insoluble particles or partially insoluble components, such as vegetable oils, mineral oils, silicone oils, solid particles, abrasives, and similar articles. The composition provides a means to include otherwise difficult to incorporate components in surfactant mixtures resulting in cosmetic preparations with multi-functional benefits including, in some cases, cleansing, moisturizing, improved skin feel, exfoliation/abrasion, novel appearance, or a combination of these benefits.

As used herein, the terminology “Non-Newtonian shear thinning viscosity” means a viscosity that decreases with an increase in shear rate. Non-Newtonian shear thinning viscosity is measured by known viscometric methods, such as for example, using a rotational viscometer such as a Brookfield viscometer. The ability of a composition to suspend water insoluble or partially water insoluble components is typically measured by mixing the composition with sufficient vigor to entrap air bubbles in the composition and then visually observing whether the air bubbles remain entrapped in the composition for a defined period of time, such as for example, 12 to 24 hours, under defined environmental conditions, such as for example, room temperature..

In some cases, the compositions of the invention may be used to suspend agents useful in skin and hair care treatments including, but not limited to, UV absorbers, hair conditioning agents, hair and skin conditioning agents for use in 2 in 1 child care formulations that are tear free, skin conditioning agents, anti-bacterial agents, styling polymers for hair and skin care formulations (including rinse off applications such as shampoos), conditioning polymers for hair and skin care formulations, precipitated conditioning polymers for enhanced active delivery to hair and skin, conditioning polymers possessing high molecular weights and/or cationic charge densities for hair and skin care formulations, surfactants usually associated with solid formulations (such as cocoyl isethionates), and swellable polymers which hydrate only on application. The compositions of the invention may also be used in the preparation of stable, multi-phase personal care formulations, including those with colored stripes found in body washes, hair shampoos, skin cleansers, child care formulations, facial washes, and skin treatments.

In some embodiments of the present invention it is desirable to include water-insoluble particles or partially insoluble components in the free flowing composition. The terms “water-insoluble particles” and “partially insoluble components” mean solid or non-solid entities which are not completely solubilized in the aqueous medium of the subject composition and include either insoluble or partially soluble species. The terms “water-insoluble particles” and “partially insoluble components” are also understood to mean and encompass those situations where the solid or non-solid entities are present at concentrations above their solubility limit and therefore portions thereof remain undissolved. Typically, the water-insoluble particles or partially insoluble components can be solid particles, liquid ingredients, gases, or mixtures thereof. Some preferred examples of gases include air bubbles. Solid particles could include, for example, solid particles of zinc pyrethione, mica, alumina, silicon pigments, moisturizing beads, natural abrasives, synthetic abrasives (exfoliants) such as polyoxyethylene beads, and apricot seeds. The water-insoluble particles typically have an average particle size from about 0.5 to about 3,000 microns in diameter. The ability to suspend water-insoluble particles or partially soluble components is a desirable feature of the free-flowing non-Newtonian shear thinning liquid composition of the present invention.

Other examples of components that may be suspended by the compositions of the present invention are a number of benefit agents. A “benefit agent” means any active ingredient that is to be delivered into the skin or hair, or onto the skin or hair, or both, at a desired location. The suspended benefit agents may be present in an amount of from about 0 to about 35% by weight of the composition.

More particularly, the suspended benefit agents may include vegetable oils, including arachis oil, castor oil, cocoa butter, coconut oil, corn oil, cotton seed oil, olive oil, palm kernel oil, rapeseed oil, safflower seed oil, sesame seed oil and soybean oil, esters, including butyl myristate, cetyl palmitate, decyloleate, glyceryl laurate, glyceryl ricinoleate, glyceryl stearate, glyceryl isostearate, hexyl laurate, isobutyl palmitate, isocetyl stearate, isopropyl isostearate, isopropyl laurate, isopropyl linoleate, isopropyl myristate, isopropyl palmitate, isopropyl stearate, propylene glycol monolaurate, propylene glycol ricinoleate, propylene glycol stearate, and propylene glycol isostearate, animal fats, including acetylated lanolin alcohols, lanolin, lard, mink oil and tallow, and fatty acids and alcohols, including behenic acid, palmitic acid, stearic acid, behenyl alcohol, cetyl alcohol, eicosanyl alcohol and isocetyl alcohol.

Other examples of suitable benefit agents include depigmentation agents, reflectants, UV absorbers, thickening agents, detangling/wet combing agents, film forming polymers, humectants, amino acids and their derivatives, antimicrobial agents, anti-acne agents, anti-aging agents, antiseptics, analgesics, local anesthetics, anti-hair loss agents, hair growth inhibitor agents, inflammation inhibitors, proteins, deodorants and anti-perspirants, agents for treatment of dandruff, seborreheic dermatitis and psoriasis, skin emollients and skin moisturizers, hair conditioners, hair softeners, hair moisturizers, vitamins, tanning agents, skin lightening agents, antifungals such as antifungals for foot preparations, depilating agents, counterirritants, hemorrhoidals, insecticides, pigments or opacifying agents, moisturizing beads, natural abrasives, synthetic abrasives such as polyoxyethylene beads, mineral oils, petrolatum, silicone oils, polyalkylsiloxanes, polyalkyarylsiloxanes, sunscreens and the like, and mixtures thereof.

Suitable reflectants include, for example, mica, alumina, calcium silicate, glycol dioleate, glycol distearate, silica, sodium magnesium fluorosilicate, and mixtures thereof.

Suitable UV absorbers include, for example, benzophenone, bornelone, PABA (Para Amino Benzoic Acid), butyl PABA, cinnamidopropyl trimethyl ammonium chloride, disodium distyrylbiphenyl disulfonate, potassium methoxycinnamate, and mixtures thereof.

Commercially available thickening agents capable of imparting the appropriate viscosity to the compositions are suitable for use in this invention.

Suitable thickening agents include, for example, mono or diesters of polyethylene glycol of the formula:
HO—(CH₂CH₂O)_{z}H (28)
wherein z is an integer from about 3 to about 200, fatty acids containing from about 16 to about 22 carbon atoms, fatty acid esters of alkoxy polyols, alkoxy derivatives of mono and diesters of fatty acids and glycerine, hydroxyalkyl cellulose, alkyl cellulose, hydroxyalkyl alkyl cellulose, and mixtures thereof. More specifically, suitable thickening agents nonexclusively include, for example, behenalkonium chloride, cetyl alcohol, quaternium 46, PG-hydroxyethyl cellulose, cocodimonium chloride, polyquaternium 6, polyquaternium 7, quaternium 18, PEG-18 glycerol oleate/cocoate, a mixture of acrylates/spirit 50 acrylate copolymer, laureth 3 and propylene glycol, a mixture of cocamidopropylbetaine and glyceryl laurate, a mixture of propylene glycol, PEG 55, and propylene glycol oleate, and mixtures thereof. Preferred thickeners include polyethylene glycol ester, and more preferably PEG-150 distearate.

Suitable detangling/wet combing agents include, for example, dioleoylamidoethyl hydroxythylmonium methosulfate, di (soyoylethyl) hydroxyethylmonium methosulfate, hydroxyethyl behenamidopropyl dimonium chloride, olealkonium chloride, polyquaternium 47, stearalkonium chloride, tricetylmonium chloride, guar hydroxypropyltrimonium chloride, hydroxypropyl guar hydroxypropyltrimonium chloride and mixtures thereof.

Suitable film forming polymers include, for example, those that, upon drying, produce a substantially continuous coating or film on the hair, skin, or nails. Examples of suitable film forming polymers include acrylamidopropyl trimonium chloride/acrylamide copolymer, corn starch/acrylamide/sodium acrylate copolymer, polyquaternium 10, polyquaternium 47, polyvinylmethyl/maleic anhydride copolymer, styrene/acrylates copolymers, and mixtures thereof.

Commercially available humectants which are capable of providing moisturization and conditioning properties to the composition are suitable for use in the present invention. The humectant is preferably present in an amount of from about 0 percent to about 10 percent, more preferably from about 0.5 percent to about 5 percent, and most preferably from about 0.5 percent to about 3 percent, based on the overall weight of the composition. Examples of suitable humectants include: water soluble liquid polyols such as glycerine, propylene glycol, hexylene glycol, butylene glycol, pentylene glycol, dipropylene glycol, and mixtures thereof, polyalkylene glycols of the formula:
HO—(R″O)_{b}—H (29)
wherein R″ is an alkylene group having from about 2 to about 4 carbon atoms and b is an integer of from about 1 to about 10 (such as PEG 4), polyethylene glycol ethers of methyl glucose having the formula:
CH₃—C₆H₁₀O₅—(OCH₂CH₂)_{c}—OH (30)
wherein c is an integer from about 5 to about 25, urea, fructose, glucose, honey, lactic acid, maltose, sodium glucuronate, and mixtures thereof. In a more preferred embodiment, the humectant is glycerine.

Suitable amino acids which may be beneficial to hair and skin and in some cases can be included as conditioning agents in the compositions of the present invention include amino acids derived from the hydrolysis of various proteins as well as the salts, esters, and acyl derivatives thereof. Examples of such amino acids nonexclusively include amphoteric and/or zwitterionic amino acids such as alkylamido alkylamines, stearyl acetyl glutamate, capryloyl silk amino acids, capryloyl collagen amino acids, capryloyl keratin amino acids, capryloyl pea amino acids, cocodimonium hydroxypropyl silk amino acids, corn gluten amino acids, cysteine, hair keratin amino acids, hair amino acids such as aspartic acid, threonine, serine, glutamic acid, proline, glycine, alanine, half-cystine, valine, methionine, isoleucine, leucine, tyrosine, phenylalanine, cysteic acid, lysine, histidine, arginine, cysteine, tryptophan, citrulline, other silk amino acids and wheat amino acids, and mixtures thereof.

Suitable proteins which may be beneficial to hair and skin and in some cases can be included as conditioning agents include those polymers that have a long chain, i.e. at least about 10 carbon atoms, and a high molecular weight, i.e. at least about 1000, and are formed by self-condensation of amino acids. Examples of such proteins include collagen, deoxyribonuclease, iodized corn protein, keratin, milk protein, protease, serum protein, silk, sweet almond protein, wheat germ protein, wheat protein, alpha and beta helix of keratin proteins, hair proteins such as intermediate filament proteins, high-sulfur proteins, ultrahigh-sulfur proteins, intermediate filament-associated proteins, high-tyrosine proteins, high-glycine tyrosine proteins, tricohyalin, and mixtures thereof.

Suitable vitamins which may be beneficial to hair and skin and in some cases can be included as conditioning agents include vitamin B complex, including thiamine, nicotinic acid, biotin, pantothenic acid, choline, riboflavin, vitamin B6, vitamin B12, pyridoxine, inositol, camitine, vitamins A,C,D,E,K and their derivatives, such as vitamin A palmitate, and pro-vitamins, e.g., panthenol (pro vitamin B5), panthenol triacetate and mixtures thereof.

Suitable antibacterial agents for hair and skin care applications include bacitracin, erythromycin, triclosan, neomycin, tetracycline, chlortetracycline, benzethonium chloride, phenol, parachlorometa xylenol (PCMX), triclocarban (TCC), chlorhexidine gluconate (CHG), zinc pyrithione, selenium sulfide and mixtures thereof.

Suitable skin emollients and skin moisturizers include, for example, vegetable oils such as arachis oil, castor oil, cocoa butter, coconut oil, corn oil, cotton seed oil, olive oil, palm kernel oil, rapeseed oil, safflower seed oil, sesame seed oil and soybean oil, esters such as butyl myristate, cetyl palmitate, decyloleate, glyceryl laurate, glyceryl ricinoleate, glyceryl stearate, glyceryl isostearate, hexyl laurate, isobutyl palmitate, isocetyl stearate, isopropyl isostearate, isopropyl laurate, isopropyl linoleate, isopropyl myristate, isopropyl palmitate, isopropyl stearate, propylene glycol monolaurate, propylene glycol ricinoleate, propylene glycol stearate, and propylene glycol isostearate, animal fats such as acetylated lanolin alcohols, lanolin, lard, mink oil and tallow, fatty acids and alcohols of behenic acid, palmitic acid, stearic acid, behenyl alcohol, cetyl alcohol, eicosanyl alcohol and isocetyl alcohol.

Additional skin treatment agents and skin conditioning agents include salicylic acid, alpha hydroxy acids, vitamins, vitamin complexes, abrasives, silicones, silicone derivatives, polymers, natural oils, synthetic oils, mineral oils, lanolin, vegetable oils, isostearyl isostearate, glyceryl laurate, methyl gluceth 10, methyl gluceth 20, chitosan, and mixtures thereof.

Suitable hair conditioners include, for example, silicones, silicone derivatives, natural oils, synthetic oils, nonionic surfactants, cationic surfactants, waxes, and polymers. Quaternized compounds such as behenamidopropyl PG-dimonium chloride, tricetylammonium chloride, dihydrogenated tallowamidoethyl hydroxyethylmonium methosulfate, and mixtures thereof, as well as lipophilic compounds like cetyl alcohol, stearyl alcohol, hydrogenated polydecene, and mixtures thereof, may also be used.

Suitable hair conditioning polymers include, for example, natural and/or synthetic cationic polymers, e.g. quaternized guar, quaternized cellulose, polyquaternium-7 and similar polymers typically at concentrations from about 0.1% to about 3.0% by weight of said composition, natural and/or synthetic nonionic polymers such as alkoxy or propoxylated guar or cellulose, alkyl guar or cellulose, polyethylene glycol, or a mixture of natural and synthetic nonionic polymers typically at concentrations from about 0.1% to about 3.0% by weight of said composition, and polyhydrol moisturizing agents, e.g. glycerine, propylene glycol, sorbitol and similar polymers. Preferable concentrations of polyhydrol moisturizing agents are typically in the range of about 0.2% to about 0.5% by weight of the composition.

Suitable hair softeners include, for example, silicone compounds, such as those that are either non-volatile or volatile, or mixtures thereof, and those that are water soluble or water insoluble, or mixtures thereof. Examples of suitable silicones include organo-substituted polysiloxanes, which are either linear or cyclic polymers of silicone/oxygen monomers and which include cetyl dimethicone, cetyl triethylammonium dimethicone copolyol phthalate, cyclomethicone, dimethicone copolyol, dimethicone copolyol lactate, hydrolyzed soy protein/dimethicone copolyol acetate, silicone quaternium 13, stearalkonium dimethicone copolyol phthalate, stearamidopropyl dimethicone, and mixtures thereof.

Suitable hair moisturizers include, for example, panthenyl ethyl ether, phytantriol, and mixtures thereof.

Suitable sunscreen agents include, for example, butyl methoxydibenzoylmethane, octyl methoxycinnamate, oxybenzone, octocrylene, octyl salicylate, phenylbenzimidazole sulfonic acid, ethyl hydroxypropyl aminobenzoate, menthyl anthranilate, aminobenzoic acid, cinoxate, diethanolamine methoxycinnamate, glyceryl aminobenzoate, titanium dioxide, zinc oxide, oxybenzone, octyl dimethyl PABA (padimate O), red petrolatum, and mixtures thereof.

Suitable tanning agents include, for example, dihydroxyacetone.

Suitableskin lightening agents include, for example, hydroquinone, catechol and its derivatives, ascorbic acid and its derivatives, and mixtures thereof.

Suitable insecticides, include, for example, insect repellents, anti-scabies and anti-lice treatments, are permethrin, pyrethrin, piperonyl butoxide, imidacloprid, N,N-diethyl toluamide, which refers to the material containing predominantly the meta isomer, i.e., N,N-diethyl-m-toluamide, which is also known as DEET, compounds of the formula: 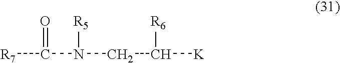
wherein R₅ is a branched or unbranched alkyl group having from about 1 to about 6 carbon atoms, R₆ is H, methyl or ethyl, R₇ is a branched or unbranched alkyl or alkoxy group having from about 1 to about 8 carbon atoms, and K is a —CN or a —COOR₈ group, wherein R₈ is a branched or unbranched alkyl group having from about 1 to about 6 carbon atoms, natural or synthetic pyrethroids, whereby the natural pyrethroids are contained in pyrethrum, the extract of the ground flowers of Chrysanthemum cinerariaefolium or Chrysanthenum coccineum, and mixtures thereof. Within the structure of formula (31) are ethyl 3-(N-butylacetamido) propionate, wherein R₇ is a CH₃ group, R₅ is an n-butyl group, R₆ is H, K is COOR₈ and R₈ is ethyl.

Suitable antifungal for foot preparations include, for example, tolnaftate.

Suitable depilating agents include, for example, calcium thioglycolate, magnesium thioglycolate, potassium thioglycolate, strontium thioglycolate, and mixtures thereof.

Suitable external analgesics and local anesthetics include, for example, benzocaine, dibucaine, benzyl alcohol, camphor, capsaicin, capsicum, capsicum oleoresin, juniper tar, menthol, methyl nicotinate, methyl salicylate, phenol, resorcinol, turpentine oil, and mixtures thereof.

Suitable antiperspirants and deodorants include, for example, aluminum chlorohydrates, aluminum zirconium chlorohydrates, and mixtures thereof.

Suitable counterirritants include, for example, camphor, menthol, methyl salicylate, peppermint oils, clove oils, ichtammol, and mixtures thereof.

Suitable inflammation inhibitors include, for example, hydrocortisone.

sSuitable hemorrhoidal products include, for example, anesthetics such as benzocaine, pramoxine hydrochloride, and mixtures thereof, antiseptics such as benzethonium chloride, astringents such as zinc oxide, bismuth subgallate, balsam Peru, and mixtures thereof, skin protectants such as cod liver oil, vegetable oil, and mixtures thereof.

Suitable benefit agents having therapeutic components that are effective in the treatment of dandruff, seborrheic dermatitis, and psoriasis, as well as the symptoms associated therewith, include, for example, zinc pyrithione, shale oil and derivatives thereof such as sulfonated shale oil, selenium sulfide, sulfur, salicylic acid, coal tar, povidone-iodine, imidazoles such as ketoconazole, dichlorophenyl imidazolodioxalan, clotrimazole, itraconazole, miconazole, climbazole, tioconazole, sulconazole, butoconazole, fluconazole, miconazolenitrite and any possible stereo isomers and derivatives thereof such as anthralin, piroctone olamine (Octopirox), selenium sulfide, ciclopirox olamine, anti-psoriasis agents such as vitamin D analogs, e.g. calcipotriol, calcitriol, and tacaleitrol, vitamin A analogs such as esters of vitamin A including vitamin A palmitate, retinoids, retinols, and retinoic acid, corticosteroids such as hydrocortisone, clobetasone, butyrate, clobetasol propionate, and mixtures thereof.

Some preferred benefit agents for treatment of dandruff, seborrheic dermatitis, and psoriasis, as well as the symptoms associated therewith, include sulfonated shale oil, elubiol, 6-(1-piperidinyl)-2-4-pyrimidinediamine-3-oxide, finasteride, ketoconazole, salicylic acid, zinc pyrithione, coal tar, benzoyl peroxide, selenium sulfide, hydrocortisone, sulfur, menthol, praxomine hydrochloride, tricetylammonium chloride, polyquaternium 10, panthenol, panthenol triacetate, vitamin A and derivatives thereof, vitamin B and derivatives thereof, vitamin C and derivatives thereof, vitamin D and derivatives thereof, vitamin E and derivatives thereof, vitamin K and derivatives thereof, keratin, lysine, arginine, hydrolzed wheat proteins, hydrolyzed silk proteins, octyl methoxycinnamate, oxybenzone, minoxidil, titanium dioxide, zinc dioxide, retinol, erythromycin, tretinoin, and mixtures thereof.

Benefit agents suitable for treating hair loss include, for example, potassium channel openers or peripheral vasodilators such as minoxidil, diazoxide, and compounds such as N″-cyano-N-(tert-pentyl)-N′-3-pyridinyl-guanidine (“P-1075”) as disclosed in U.S. Pat. No. 5,244,664, which is incorporated by reference herein, vitamins, such as vitamin E and vitamin C, and derivatives thereof such as vitamin E acetate and vitamin C palmitate, hormones such as erythropoietin, prostaglandins, such as prostaglandin EI and prostaglandin F2-alpha, fatty acids such as oleic acid, diuretics such as spironolactone, heat shock proteins (“HSP”), such as HSP 27 and HSP 72, calcium channel blockers, such as verapamil HCL, nifedipine, and diltiazemamiloride, immunosuppressant drugs, such as cyclosporin and Fk-506, 5 alpha-reductase inhibitors such as finasteride, growth factors such as EGF, IGF and FGF, transforming growth factor beta, tumor necrosis factor, non-steroidal anti-inflammatory agents such as benoxaprofen, retinoids and derivatives thereof such as tretinoin, cytokines, such as IL-6, IL-1 alpha, and IL-1 beta, cell adhesion molecules such as ICAM, glucocorticoids such as betamethasone, botanical extracts such as aloe, clove, ginseng, rehmannia, swertia, sweet orange, zanthoxylum, Serenoa repens (saw palmetto), Hypoxis rooperi, stinging nettle, pumpkin seeds, and rye pollen, other botanical extracts including sandalwood, red beet root, chrysanthemum, rosemary, burdock root and other hair growth promoter activators as disclosed in DE 4330597, which is incorporated by reference herein to the extent that it is not inconsistent with the present application, homeopathic agents such as Kalium Phosphoricum D2, Azadirachta indica D2, and Joborandi DI, genes for cytokines, growth factors, and male-pattern baldness, antifungals such as ketoconazole and elubiol, antibiotics such as streptomycin, protein inhibitors such as cycloheximide, acetazolamide, benoxaprofen, cortisone, diltiazem, hexachlorobenzene, hydantoin, nifedipine, penicillamine, phenothiazines, pinacidil, psoralens, verapamil, zidovudine, alpha-glucosylated rutin having at least one rutin selected from quercetin, isoquercitrin, hesperidin, naringin, and methylhesperidin, and flavonoids and transglycosidated derivatives thereof which are all disclosed in JP 7002677, which is incorporated by reference herein to the extent that it is not inconsistent with the present application, and mixtures thereof.

Benefit agents suitable for use in inhibiting hair growth include, for example, serine proteases such as trypsin, vitamins such as alpha-tocopherol (vitamin E) and derivatives thereof such as tocopherol acetate and tocopherol palmitate, antineoplastic agents, such as doxorubicin, cyclophosphamide, chlormethine, methotrexate, fluorouracil, vincristine, daunorubicin, bleomycin and hydroxycarbamide, anticoagulants, such as heparin, heparinoids, coumaerins, detran and indandiones, antithyroid drugs, such as iodine, thiouracils and carbimazole, lithium and lithium carbonate, interferons, such as interferon alpha, interferon alpha-2a and interferon alpha-2b, retinoids, such as retinol (vitamin A), isotretinoin, glucocorticoids such as betamethasone, and dexamethosone, antihyperlipidaemic drugs, such as triparanol and clofibrate, thallium, mercury, albendazole, allopurinol, amiodarone, amphetamines, androgens, bromocriptine, butyrophenones, carbamazepine, cholestyramine, cimetidine, clofibrate, danazol, desipramine, dixyrazine, ethambutol, etionamide, fluoxetine, gentamicin, gold salts, hydantoins, ibuprofen, imipramine, immunoglobulins, indandiones, indomethacin, intraconazole, levadopa, maprotiline, methysergide, metoprolol, metyrapone, nadolol, nicotinic acid, potassium thiocyanate, propranolol, pyridostimine, salicylates, sulfasalazine, terfenadine, thiamphenicol, thiouracils, trimethadione, troparanol, valproic acid, and mixtures thereof.

Suitable anti-aging agents include, for example, inorganic sunscreens such as titanium dioxide and zinc oxide, organic sunscreens such as octyl-methyl cinnamates and derivatives thereof, retinoids, vitamins such as vitamin E, vitamin A, vitamin C, vitamin B, and derivatives thereof such as vitamin E acetate, vitamin C palmitate, and the like, antioxidants including beta carotene, alpha hydroxy acid such as glycolic acid, citric acid, lactic acid, malic acid, mandelic acid, ascorbic acid, alpha-hydroxybutyric acid, alpha-hydroxyisobutyric acid, alpha-hydroxyisocaproic acid, atrolactic acid, alpha-hydroxyisovaleric acid, ethyl pyruvate, galacturonic acid, glucoheptonic acid, glucopheptono 1,4-lactone, gluconic acid, gluconolactone, glucuronic acid, glucuronolactone, glycolic acid, isopropyl pyruvate, methyl pyruvate, mucic acid, pyruvic acid, saccharic acid, saccharic acid 1,4-lactone, tartaric acid, and tartronic acid, beta hydroxy acids such as beta-hydroxybutyric acid, beta-phenyl-lactic acid, beta-phenylpyruvic acid, botanical extracts such as green tea, soy, milk thistle, algae, aloe, angelica, bitter orange, coffee, goldthread, grapefruit, hoellen, honeysuckle, Job's tears, lithospermum, mulberry, peony, puerarua, rice, safflower, and mixtures thereof.

Some preferred anti-aging agents comprise retinoids including retinol and tretinoin, anti-oxidants, alpha-hydroxy acids and beta-hydroxy acids.

Suitable anti-acne agents include, for example, topical retinoids including tretinoin, isotretinoin, motretinide, adapalene, tazarotene, azelaic acid, retinol, salicylic acid, benzoyl peroxide, resorcinol, antibiotics such as tetracycline and isomers thereof, erythromycin, anti-inflammatory agents such as ibuprofen, naproxen, hetprofen, botanical extracts such as alnus, amica, artemisia capillaris, asiasarum root, birth or afterbirth, calendula, chamomile, cnidium, comfrey, fennel, galla rhois, hawthorn, houttuynia, hypericum, jujube, kiwi, licorice, magnolia, olive, peppermint, philodendron, salvia, sasa albomarginata, imidazoles such as ketoconazole and elubiol, those anti-acne agents described in Gollnick, H. et al. 196(I) Dermatology Sebaceous Glands, Acne and Related Disorders, 119-157 (1998), which is incorporated by reference herein to the extent that it is not inconsistent with the present application, and mixtures thereof.

Suitable depigmentation agents include, for example, retinoids such as retinol, kojic acid and its derivatives such as, for example, kojic dipalmitate, hydroquinone and its derivatives such as arbutin, transexamic acid, vitamins such as niacin, vitamin C and its derivatives, azelaic acid, placertia, licorice, extracts such as chamomile and green tea, and mixtures thereof. Retinol, kojic acid, and hydroquinone are preferred.

Other examples of benefit agents include allergy inhibitors, anti-wrinkling agents, anti-pruritics, antitussives, hair growth promoting agents, antihistamines, anticholinergics, anti-emetics, antiinfectives, vasoconstrictors, vasodilators, wound healing promoters, peptides, polypeptides, medicament agents, shaving preparations, poison ivy products, poison oak products, burn products, anti-diaper rash agents, prickly heat agents, herbal extracts, retinal, flavoides, sensates, skin conditioners, hair lighteners, cell turnover enhancers and the like, and mixtures thereof.

Other components that may be added to the compositions include typical components added to personal care products, all of which are useful in enhancing the appearance or cosmetic properties of the product. These may include, for example, auxiliary thickeners such as carboxymethylcellulose, magnesium aluminum silicate, hydroxyethylcellulose, methylcellulose, carbopols, glucamides, sequestering agents such as tetrasodium ethylenediaminetetraacetate (Na₄-EDTA), EHDP or mixtures thereof, which can be present in varying amounts including amounts ranging from about 0.01 to about 5%, preferably about 0.01% to about 3%, and coloring agents, pigments, perfumes, opacifiers and pearlizers such as zinc stearate, magnesium stearate, TiO₂, mica, EGMS (ethylene glycol monostearate), EGDS (ethylene glycol distearate), and Lytron 621 (Styrene/Acrylate copolymer).

Inclusion of antimicrobials may be used advantageously in some embodiments. Such antimicrobials include, for example, 2-hydroxy-4,2′4′ trichlorodiphenylether (DP300), preservatives such as dimethyloldimethylhydantoin (Glydant XL1000), parabens, sorbic acid, etc., antioxidants such as, for example, butylated hydroxytoluene (BHT), and mixtures thereof.

### EXAMPLES 1-9

The compositons of Examples 1-9 are made by mixing the components in the relative amounts listed in TABLE I below.

TABLE I
(All component amounts indicate the relative amount
(by weigh)t of the component in the composition)

| Component | 1 | 2 | 3 | 4 | 5 | 6 | 7 | 8 | 9 |
|---|---|---|---|---|---|---|---|---|---|
| Sodium Trideceth-3 | 24.5 | 24.5 | — | — | — | — | — | — | — |
| Sulfate (29.41% AI) | | | | | | | | | |
| Sodium Tridecyl | — | — | 29.5 | — | — | — | — | — | — |
| Sulfate (24.40% AI) | | | | | | | | | |
| Ammonium Tridecyl | — | — | — | 27.9 | 27.9 | 27.9 | — | — | — |
| Sulfate (25.83% AI) | | | | | | | | | |
| Ammonium | — | — | — | — | — | — | 27.6 | 27.6 | 27.6 |
| Trideceth-3 Sulfate | | | | | | | | | |
| (26.13% AI) | | | | | | | | | |
| Miranol Ultra L-32 | 8.3 | 8.3 | 8.3 | 8.3 | 8.3 | 8.3 | 8.3 | 8.3 | 8.3 |
| (Sodium | | | | | | | | | |
| Lauroamphoacetate, | | | | | | | | | |
| 32% AI) | | | | | | | | | |
| Water | 27.2 | 27.2 | 22.2 | 23.8 | 23.8 | 23.8 | 24.2 | 24.2 | 24.2 |
| Citric Acid, 50% | 1.0 | 1.0 | 1.2 | 2.7 | 2.7 | 2.7 | 1.9 | 1.9 | 1.9 |
| Sodium Chloride | 4 | 5 | 1 | 1 | 2 | 3 | 4 | 5 | 6 |
| pH | 5.5 | 5.5 | 5.6 | 5.5 | 5.5 | 5.5 | 5.6 | 5.6 | 5.6 |

### Comparative Example C1 & Examples 10-12

Comparative Example C1 and Examples 10-12 were made according to the following base recipe and procedure:
1. prepare 12% (as active surfactant) aqueous mixture of anionic surfactant (chosen from Sodium trideceth-3 sulfate, Sodium tridecyl sulfate, Ammonium trideceth-3 sulfate, Ammonium tridecyl sulfate),
2. add 4.4% (as active surfactant) Sodium Lauroamphoacetate (Miranol Ultra L-32, Rhodia, 32% active solution) to the aqueous surfactant mixture,
3. adjust mixture to desired concentration with diluting with deionized water,
4. adjust pH of mixture to 5.5- 5.65 with 50% citric acid, and

5. add salt to mixture, as indicated in TABLES II - V below.

TABLE II

| Comparative Example C1: | | | |
|---|---|---|---|
| Sodium Trideceth-3 Sulfate | % | Centrifuge | Capable of |
| (Rhodapex EST 30, Rhodia, 29.4% | Sodium | Test | Suspending |
| active) | Chloride | 1-phase? | Air? |
| C1-1 | 0 | 2-Phase | No |
| C1-2 | 1 | 2-Phase | No |
| C1-3 | 2 | 2-Phase | No |
| C1-4 | 3 | 2-Phase | No |
| C1-5 | 4 | 1-Phase | Yes |
| C1-6 | 5 | 1-Phase | Yes |
| C1-7 | 6 | 1-Phase | Yes |

TABLE III

| Example 10: | | | |
|---|---|---|---|
| Sodium Tridecyl Sulfate | % | Centrifuge | Capable of |
| (Rhodapon TDS, Rhodia, 24.4% | Sodium | Test 1- | Suspending |
| active) | Chloride | phase? | Air? |
| 10-1 | 0 | 2-Phase | No |
| 10-2 | 1 | 1-Phase | Yes |
| 10-3 | 2 | 2-Phase | No |

TABLE IV

| Example 11: | % | Centrifuge | Capable of |
|---|---|---|---|
| Ammonium Trideceth-3 Sulfate | Sodium | Test | Suspending |
| (Rhodia, 26.1% active solution) | Chloride | 1-phase? | Air? |
| 11-1 | 0 | 2-Phase | No |
| 11-2 | 1 | 2-Phase | No |
| 11-3 | 2 | 2-Phase | No |
| 11-4 | 3 | 1-Phase | No |
| 11-5 | 4 | 1-Phase | Yes |
| 11-6 | 5 | 1-Phase | Yes |
| 11-7 | 6 | 1-Phase | Yes |

TABLE V

| Example 12: | % | Centrifuge | Capable of |
|---|---|---|---|
| Ammonium Tridecyl Sulfate | Sodium | Test | Suspending |
| (Rhodia, 25.8% active solution) | Chloride | 1-phase? | Air? |
| 12-1 | 0 | 1-Phase | Yes |
| 12-2 | 1 | 1-Phase | Yes |
| 12-3 | 2 | 1-Phase | Yes |
| 12-4 | 3 | 1-Phase | No |
| 12-5 | 4 | 1-Phase | No |

The compositions of Comparative Example C1 and Examples 10-12 were subjected to centrifuging (2 milliliter sample at 20,000 G in a Marathon Model 26 KM centrifuge for 15 minutes). The centrifuged compositions were then visually examined to determine whether the compositions remained as a single, apparently homogeneous, liquid phase or had separated into two separate liquid phases. Results are noted in TABLES II-V.

The compositions of Comparative Example C1 and Examples 10-12 were mixed to incorporate air in the compositions and then subjected to allowed to sit overnight at room temperature. The next day, that is, about 12-24 hour after mixing, the compositions were visually examined at room temperature under ambient lighting to determine whether air bubbles remained suspended in the compositions. Compositions in which the air bubbles remained entrapped were evaluated as capable of suspending air. Compositions in which the air bubbles did not remain entrapped were evaluated as not being capable of suspending air. Results are noted in TABLES II-V.

Comparative Example C1 (Sodium Trideceth Sulfate) requires 4% NaCl to reach a 1-phase system that is capable of suspending. By reducing the moles of ethoxylation we see a significant reduction in the amount of structurant needed to form a 1-phase system. Example 10 only requires 1% NaCl to form a 1-phase system & Example 12 is capable of forming a 1-phase system with 0% NaCl added. (While not to be bound by the theory, it is believed that the salt coming in as a byproduct of the amphoacetate as well as the cationic nature of the amphoacetate at the pH of 5.5 will contribute to the structuring of the product.) In all examples above (except the ammonium tridecyl sulfate), additional structurant is needed to create a 1-phase system capable of suspending.

Surprisingly, as well as being a better surfactant for creating the 1-phase suspending systems, the ammonium cation also allows for a wider stability range in formulated systems. This is beneficial because accuracy of salt additions do not need to be as carefully controlled during the manufacturing process.

### Comparative Example C2 & Examples 13-15

Comparative Example C2 and Examples 13-15 were made according to the following base recipe and procedure,
1. prepare 17.5% (as active surfactant) aqueous mixture of anionic surfactant (chosen from Sodium trideceth-3 sulfate, Sodium tridecyl sulfate, Ammonium trideceth-3 sulfate, Ammonium tridecyl sulfate),
2. add 0.2% Glydant (DMDM Hydantoin, Lonza) to the mixture
3. adjust mixture to desired concentration by diluting with deionized water,
4. adjust of mixture pH to 5.6-5.7 with 50% citric acid, and

5. add cetrimonium bromide (Rhodiquat M242B/99, Rhodia, powder) to mixture, as indicated in TABLES VI-IX below.

TABLE VI

| Comparative Example C2: | | | |
|---|---|---|---|
| Sodium Trideceth-3 Sulfate | % | Centrifuge | Capable of |
| (Rhodapex EST 30, Rhodia, | Cetrimonium | Test | Suspending |
| 29.4% active) | Bromide | 1-phase? | Air? |
| C2-1 | 1 | 1-Phase | No |
| C2-2 | 2 | 1-Phase | No |
| C2-3 | 3 | 2-Phase | No |
| C2-4 | 4 | 2-Phase | No |
| C2-5 | 5 | 2-Phase | No |
| C2-6 | 6 | 1-Phase | Partial |
| C2-7 | 7 | 1-Phase | Partial |
| C2-8 | 8 | 1-Phase | Yes |
| C2-9 | 9 | 1-Phase | Yes |

TABLE VII

| Example 13: | | | |
|---|---|---|---|
| Sodium Tridecyl Sulfate | % | Centrifuge | Capable of |
| (Rhodapon TDS, Rhodia, 24.4% | Cetrimonium | Test | Suspending |
| active) | Bromide | 1-phase? | Air? |
| 13-1 | 1 | 1-Phase | No |
| 13-2 | 2 | 2-Phase | No |
| 13-3 | 3 | 2-Phase | No |
| 13-4 | 4 | 2-Phase | No |
| 13-5 | 5 | 1-Phase | Yes |
| 13-6 | 6 | 1-Phase | Yes |
| 13-7 | 7 | 1-Phase | Yes |
| 13-8 | 8 | 1-Phase | Yes |
| 13-9 | 9 | 1-Phase | Yes |

TABLE VIII

| Example 14: | % | Centrifuge | Capable of |
|---|---|---|---|
| Ammonium Trideceth-3 Sulfate | Cetrimonium | Test | Suspending |
| (Rhodia, 28.1% active) | Bromide | 1-phase? | Air? |
| 14-1 | 1 | 1-Phase | No |
| 14-2 | 2 | 2-Phase | No |
| 14-3 | 3 | 2-Phase | No |
| 14-4 | 4 | 2-Phase | No |
| 14-5 | 5 | 1-Phase | Yes |
| 14-6 | 6 | 1-Phase | Yes |
| 14-7 | 7 | 1-Phase | Yes |
| 14-8 | 8 | 1-Phase | Yes |
| 14-9 | 9 | 1-Phase | Yes |

TABLE IX

| Example 15: | % | Centrifuge | Capable of |
|---|---|---|---|
| Ammonium Tridecyl Sulfate | Cetrimonium | Test | Suspending |
| (Rhodia, 29.0% active) | Bromide | 1-phase? | Air? |
| 15-1 | 1 | 2-Phase | No |
| 15-2 | 2 | 1-Phase | Yes |
| 15-3 | 3 | 1-Phase | Yes |
| 15-4 | 4 | 1-Phase | Yes |
| 15-5 | 5 | 1-Phase | Yes |
| 15-6 | 6 | 1-Phase | Yes |
| 15-7 | 7 | 1-Phase | Yes |
| 15-8 | 8 | 1-Phase | Yes |
| 15-9 | 9 | 1-Phase | Yes |

The compositions of Comparative Example C2 and Examples 13-15 were evaluated for phase separation and air suspending ability using the methods described above in reference to Comparative Example C1 and Examples 10-12. Results are set forth above in the TABLES VI-IX.

Using Cetrimonium Bromide as the structure inducing agent, the Comparative Example C2 (Sodium Trideceth Sulfate) 8% CETAB to create a 1-phase system capable of suspending. By reducing the moles of ethoxylation we are able to reduce the amount of structuring agent to 5% in Example 13 (Sodium Tridecyl Sulfate) and 2% in Example 6 (Ammonium Tridecyl Sulfate). Additionally, the ammonium cation is better than the sodium cation for creating 1-phase systems capable of suspending. Example 14 (ammonium trideceth sulfate) only requires 5% of the CETAB to make a 1-phase suspending system while the comparative example (sodium trideceth sulfate) required 8%. Comparing Example 15 and Example 13 (sodium vs. ammonium) the amount of structuring agent is reduced by over half when using the ammonium cation. By reducing the amount of cetrimonium bromide needed in the formulation, there is significant cost savings to the manufacturer and consumer.

### Comparative Example C3 & Examples 16-17

Comparative Example C3 and Examples 16-17 listed below were all made with the following base recipe and procedure:
1. prepare 14.2% (as active surfactant) aqueous mixture of anionic surfactant (chosen from Sodium trideceth-3 sulfate, Sodium tridecyl sulfate, Ammonium trideceth-3 sulfate, Ammonium tridecyl sulfate),
2. add 4.7% (as active surfactant) Sodium Lauroamphoacetate (Miranol Ultra L-32, Rhodia, 32% active solution) to mixture,
3. add 1.4% Cocamide MEA (Alkamide C-212, Rhodia, flakes) to mixture,
4. add 1.7% (as active surfactant) Laureth-7 (Rhodasurf L-7/90, Rhodia, 90% active solution) to mixture,
5. add 0.1 % Glydant to mixture,
6. adjust mixture to desired concentration by diluting with deionized water,
7. adjust mixture pH to 5.5-5.65 with 50% citric acid, and

8. add salt as indicated in TABLES X-XII below.

TABLE X

| Comparative Example C3: | | | |
|---|---|---|---|
| Sodium Trideceth-3 Sulfate | | Centrifuge | Capable of |
| (Rhodapex EST 30, Rhodia, | % Sodium | Test | Suspending |
| 29.4% active) | Chloride | 1-phase? | Air? |
| C3-1 | 0 | 2-Phase | No |
| C3-2 | 1.5 | 2-Phase | No |
| C3-3 | 3 | 1-Phase | No |
| C3-4 | 4.4 | 1-Phase | Yes |

TABLE XI

| Example 16: | | | |
|---|---|---|---|
| Sodium Tridecyl Sulfate | | Centrifuge | Capable of |
| (Rhodapon TDS, Rhodia, 24.4% | % Sodium | Test | Suspending |
| active) | Chloride | 1-phase? | Air? |
| 16-1 | 0 | 2-Phase | No |
| 16-2 | 1.5 | 1-Phase | Yes |
| 16-3 | 3 | 1-Phase | Yes |
| 16-4 | 4.4 | 1-Phase | Yes |

TABLE XII

| Example 17: | | Centrifuge | Capable of |
|---|---|---|---|
| Ammonium Tridecyl Sulfate | % Sodium | Test | Suspending |
| (Rhodia, 29.0% active) | Chloride | 1-phase? | Air? |
| 17-1 | 0 | 1-Phase | Yes |
| 17-2 | 1.5 | 1-Phase | Yes |
| 17-3 | 3 | 1-Phase | Yes |
| 17-4 | 4.4 | 1-Phase | Yes |

The compositions of Comparative Example C3 and Examples 16 and 17 were evaluated for phase separation and air suspending ability using the methods described above in reference to Comparative Example C1 and Examples 10-12. Results are set forth above in the TABLES X-XII.

Comparative Example C3 (Sodium Trideceth-3 Sulfate) reaches a 1-phase system capable of suspending by using 4.4% sodium chloride. Reducing the moles of ethoxylation in Example 16 (Sodium Tridecyl Sulfate) allows for a 1-phase system capable of suspending with significantly less structurant, 1.5% sodium chloride. The amount of additional structuring agent (sodium chloride) needed in Example 17 (Ammonium Tridecyl Sulfate which has the ammonium cation and a lower moles of ethoxylation) is significantly reduced to make a 1-phase suspending system.

An exemplary method for using the compositions of Examples 1 to 17 for preparing selected exemplary composition a desired amount by adding water to the composition. It is preferable to add polymers at this point to ensure easy dispersion, however the polymers may be added later in the formulation if desired and if there are no dispersion problems. In particular, cationic polymers such as cationic guar gums, may be added initially to the water under moderate to high stirring or they may be pre-solubilized in glycerin and added later in the process. Follow any process instructions for the given polymer to ensure proper hydration and/or dissolution. Add all of the surface active agents (i.e. surfactants) to the water with moderate agitation while stirring. When solid surfactants are used, the mixtures are heated to a minimum of about 5-10° C. above the melting temperature of the solid surfactant. The mixtures are stirred until they become homogenous and, when heating is used, stirring is continued until the mixture is cooled to ambient temperature. The pH is then adjusted to about 5.0 to 6.5 and solid benefit agent and the electrolyte are added with stirring to disperse.

An exemplary method for using the compositions of Examples 1 to 17 for preparing formulations that can incorporate emollients into formulations is as follows. Dilute the selected exemplary composition a desired amount by adding water to the composition. It is preferable to add polymers at this point to ensure easy dispersion, however the polymers may be added later in the formulation if desired and if there are no dispersion problems. In particular, cationic polymers such as cationic guar gums, may be added initially to the water under moderate to high stirring or they may be pre-solubilized in glycerin and added later in the process. Follow any process instructions for the given polymer to ensure proper hydration and/or dissolution. Add all of the surface active agents (i.e. surfactants) to the water with moderate agitation while stirring. When solid surfactants are used, the mixtures are heated to a minimum of about 5-10° C. above the melting temperature of the solid surfactant. It is typical for the emollient to be added at this point. The mixtures are stirred until they become homogenous and, when heating is used, stirring is continued until the mixture is cooled to ambient temperature. The pH is typically adjusted with citric acid to about 5.0 to 6.5, and heat sensitive additives such as color, fragrance, and preservatives, for example, as well as the electrolyte, can be added. Mixing may be continued for 1-2 hours after the addition of electrolyte.

Those persons skilled in the art will appreciate that the present invention is susceptible to a broad utility and application. Many embodiments and adaptations of the invention, including various methods for preparing the composition of the present invention other than those herein described, as well as many variations and modifications, will be apparent from or reasonably suggested by the present invention and the foregoing description thereof, without departing from the substance or scope of the present invention. Accordingly, while the present invention has been described herein in detail in exemplary embodiments, it is to be understood that this disclosure is only illustrative and exemplary of the present invention and is made merely for purposes of providing a full and enabling disclosure of the invention. The foregoing is not intended or to be construed to limit the present invention or otherwise to exclude any such other embodiments, adaptations, variations, modifications, the present invention being limited only by the claims appended hereto and the equivalents thereof.

1. A structured surfactant composition, comprising:
one or more branched alkyl (ether) sulfates according to the formula:
RO(CH₂CH₂O)ₙSO₃M
wherein:
R is branched (C₈-C₁₈)alkyl or branched (C₈-C₁₈)alkenyl,
n has an average value of from 0 to about 7 and
M is a solubilizing cation, provided that M cannot be sodium if n is greater than or equal to 1, and
a structurant,
wherein the composition exhibits non-Newtonian shear thinning viscosity and is capable of suspending insoluble or partially insoluble components.

2. The composition of claim 1, wherein is R is a branched (C₁₂-C₁₈)alkyl or branched (C₁₂-C₁₈)alkenyl.

3. The composition of claim 1, wherein n is from 0 to less than 1.

4. The composition of claim 1, wherein M is selected from sodium, magnesium, potassium, ammonium, and substituted ammonium.

5. The composition of claim 1, wherein M is selected from ammonium or substitued ammonium.

6. The composition of claim 1, wherein the branched alkyl ether sulfate comprises one or more of ammonium trideceth sulfate and ammonium tridecyl sulfate.

7. The composition of claim 1, wherein the branched alkyl sulfate comprises sodium tridecyl sulfate.

8. The composition of claim 1, wherein the structurant comprises a compound selected from electrolytes, alkanolamides, cationic surfactants, fatty alcohols, fatty acids and fatty acid esters.

9. The composition of claim 1, wherein the structurant comprises an electrolyte.

10. The composition of claim 1, wherein the structurant comprises an alkanolamide.

11. The composition of claim 1, wherein the structurant comprises one or more of fatty acids and fatty acid esters.

12. The composition of claim 1 further comprising at least one additional surfactant selected from the group consisting of nonionic, amphoteric, zwitterionic, and cationic surfactants.

13. The composition of claim 12, wherein the additional surfactant comprises one or more surfactant selected from amphoteric surfactants and nonionic surfactants.

14. The composition of claim 13 wherein the nonionic surfactant comprises a sorbitan derivative.

15. The composition of claim 13, wherein the-nonionic surfactant comprises a polyethylene glycol distearate.

16. The composition of claim 13, wherein the amphoteric surfactant comprises an amphoacetate compound.

17. The composition of claim 1, further comprising at least one compound selected from the group consisting essentially of water-insoluble particles, partially insoluble components, and benefit agents.

18. A personal care composition comprising the composition of claim 1.

19. The composition of claim 18, wherein the personal care formulations are body washes, shampoos, baby cleansing products, facial cleansers, hand soaps, and skin cleansers.

20. The composition of claim 18, further comprising at least one additional surfactant selected from the group consisting of nonionic, amphoteric, zwitterionic, and cationic surfactants.

21. The composition of claim 20, wherein the additional surfactant comprises one or more surfactant selected from amphoteric surfactants and nonionic surfactants.

22. The composition of claim 21, wherein the nonionic surfactant comprises a sorbitan derivative.

23. The composition of claim 21, wherein one of the nonionic surfactants included is a polyethylene glycol distearate.

24. The composition of claim 21, wherein the amphoteric surfactant comprises one or more surfactants selected from amphoacetate amphoteric surfactants, betaine amphoteric surfactants, sultaine amphoteric surfactants, and propionate amphoteric surfactants.

25. The composition of claim 18, further comprising at least one compound selected from the group consisting essentially of water-insoluble particles, partially insoluble components, and benefit agents.

26. An aqueous surfactant composition, comprising, based on 100 parts by weight of the composition:
from about 3 to about 50 parts by weight of one or more branched alkyl (ether) sulfates according to the formula:
RO(CH₂CH₂O)ₙSO₃M
wherein:
R is a branched (C₈-C₁₈)alkyl or branched (C₈-C₁₈)alkenyl,
n has an average value of from about 0 to about 7 and
M is a solubilizing cation, provided that M cannot be sodium if n is greater than or equal to 1, and
from about 0.1 to about 20 parts by weight of one or more structurants selected from electrolytes, cationic surfactants, and nonionic surfactants.
